SEQUENCE LISTING (19 sequences)

SEQ ID NO: 1 - DNA, 92 bp, synthetic construct
  source 1..92: mol_type = other DNA
agcctcgtctgttctccccgctaacgaactcaaacccggggcccgacgcgacatatcagctaagagtaggccggggaaagacaagcagacgt

SEQ ID NO: 2 - DNA, 92 bp, synthetic construct
  source 1..92: mol_type = other DNA
agcctcgtctgttctcccctaatcagcttcaacgagccgtacaggtggcacctcaggaggggcccacagggaggggaaagacaagcagacgt

SEQ ID NO: 3 - DNA, 16 bp, synthetic construct
  source 1..16: mol_type = other DNA
tgcttgtctttcccct

SEQ ID NO: 4 - DNA, 16 bp, synthetic construct
  source 1..16: mol_type = other DNA
ccctgtgggcccctcc

SEQ ID NO: 5 - DNA, 16 bp, synthetic construct
  source 1..16: mol_type = other DNA
tcgttgaagctgatta

SEQ ID NO: 6 - DNA, 16 bp, synthetic construct
  source 1..16: mol_type = other DNA
cggcctactcttagct

SEQ ID NO: 7 - DNA, 16 bp, synthetic construct
  source 1..16: mol_type = other DNA
gggtttgagttcgtta

SEQ ID NO: 8 - DNA, 33 bp, synthetic construct
  source 1..33: mol_type = other DNA
ctttcgcggggagaacagacgaggcttaacgtc

SEQ ID NO: 9 - DNA, 34 bp, synthetic construct
  source 1..34: mol_type = other DNA
gcggggagaacagacgaggcttaacgtctgttgt

SEQ ID NO: 10 - DNA, 40 bp, synthetic construct
  source 1..40: mol_type = other DNA
tgaggtgccacctgtacggcgatatgtcgcgtcgggcccc

SEQ ID NO: 11 - DNA, 16 bp, synthetic construct
  source 1..16: mol_type = other DNA
  modified_base 11: mod_base = OTHER; note = 5-ethynyl-2'-deoxyuridine
tgcttgtctttcccct

SEQ ID NO: 12 - DNA, 16 bp, synthetic construct
  source 1..16: mol_type = other DNA
  modified_base 6: mod_base = OTHER; note = 5-ethynyl-2'-deoxyuridine
ccctgtgggcccctcc

SEQ ID NO: 13 - DNA, 16 bp, synthetic construct
  source 1..16: mol_type = other DNA
  modified_base 11: mod_base = OTHER; note = 5-ethynyl-2'-deoxyuridine
tcgttgaagctgatta

SEQ ID NO: 14 - DNA, 16 bp, synthetic construct
  source 1..16: mol_type = other DNA
  modified_base 6: mod_base = OTHER; note = 5-ethynyl-2'-deoxyuridine
cggcctactcttagct

SEQ ID NO: 15 - DNA, 16 bp, synthetic construct
  source 1..16: mol_type = other DNA
  modified_base 11: mod_base = OTHER; note = 5-ethynyl-2'-deoxyuridine
gggtttgagttcgtta

SEQ ID NO: 16 - DNA, 92 bp, synthetic construct
  source 1..92: mol_type = other DNA
agcctcgtctgttctccccgctaacgaactcaaacccggggcccgacgcgacatatcagctaagagtaggccggggaaagctaggaccaaag

SEQ ID NO: 17 - DNA, 92 bp, synthetic construct
  source 1..92: mol_type = other DNA
ggtcacttagcgaactctgtaatcagcttcaacgagccgtacaggtggcacctcaggaggggcccacagggaggggaaagacaagcagtgac

SEQ ID NO: 18 - DNA, 33 bp, synthetic construct
  source 1..33: mol_type = other DNA
ctttccccagagttcgctaagtgacctagtcac

SEQ ID NO: 19 - DNA, 35 bp, synthetic construct
  source 1..35: mol_type = other DNA
gcggggagaacagacgaggcttactttggtcctag

(19) United States
(12) Patent Application Publication
(10) Pub. No.: US 2024/0052414 A1
(43) Pub. Date: Feb. 15, 2024

(54) NUCLEIC ACID NANOSTRUCTURES

(71) Applicant: Government of the United States of America, as represented by the Secretary of Commerce, Gaithersburg, MD (US)

(72) Inventors: Veronika Ann Szalai, Monrovia, MD (US); Jacob Michael Majikes, Gaithersburg, MD (US)

(21) Appl. No.: 18/198,457

(22) Filed: May 17, 2023

Related U.S. Application Data
(60) Provisional application No. 63/342,650, filed on May 17, 2022.

(51) Int. Cl. C12Q 1/6876; C12Q 1/6811

(52) U.S. Cl. CPC C12Q 1/6876; C12Q 1/6811

(57) ABSTRACT

Embodiments of the present invention relate to methods and composition of a nucleic acid tile with addressable locations for placement of molecular species that can function as sensors, reporters, or enhancers of measurement systems. Embodiments of the present invention may also include a nucleic acid tile with addressable locations for placement of molecular species such that attachment chemistry restricts motion of the probes and permits mixing and matching of probes for multiplexed sensing and detection.

### CROSS-REFERENCE TO RELATED APPLICATION

This application claims the benefit of priority from U.S. Provisional Patent Application Ser. No. 63/342,650, filed on May 17, 2022, the disclosure of which is incorporated herein by reference in its entirety.

### STATEMENT REGARDING FEDERAL RIGHTS

The invention described herein was made with United States Government support from the National Institute of Standards and Technology (NIST), an agency of the United States Department of Commerce. The United States Government has certain rights in the invention.

### SEQUENCE LISTING

This application includes a Sequence Listing which has been electronically submitted in XML format and is hereby incorporated by reference in its entirety. The Sequence Listing, created on Aug. 9, 2023, is named “18198457_Sequence_Listing.xml” and is 20,416 bytes in size.

### FIELD OF THE INVENTION

The present invention relates generally to nucleic acid-based nanostructures and related methods, and more particularly, to nucleic acid-based tiles with addressable locations for placement of molecular probes.

### BACKGROUND OF THE INVENTION

Organizing molecular probes on DNA origami requires DNA components that are not interchangeable, namely a single-stranded plasmid template and several small DNA oligonucleotides (staple strands). Molecular probes organized at specific sites on DNA origami-based nanostructures can be used to sense a wide variety of analytes and physical processes. Biomolecule-based “rulers” that contain more than two covalently attached probes designed for a variety of measurement modalities (e.g., fluorescence & magnetic resonance spectroscopies) are typically fabricated either via complex chemical synthesis or molecular biology methods requiring specific expertise.

The sequence specificity of a DNA tile provides for the incorporation of molecular probes at defined positions in the structure, reduces costs, and increases final concentrations compared to use of a DNA origami-based system. A DNA tile platform acts like DNA origami by providing spatial control of DNA nanofabrication and does so at a lower molar cost. Unlike DNA origami, which typically has dimensions of about 100 nm, a DNA tile is approximately 10 nm in size, which reduces the amount of commercially synthesized DNA required to produce the nanostructure and, thereby, the cost for scale-up.

Accordingly, there is a need for a DNA tile with addressable locations for placement of molecular species that can function as sensors, reporters, or enhancers of measurement systems. There is also a need for a DNA tile with addressable locations for placement of molecular species such that attachment chemistry restricts motion of the probes and permits mixing and matching of probes for multiplexed sensing and detection.

### SUMMARY OF THE INVENTION

Embodiments of the present invention relate to compositions and methods of making nucleic acid tile structures with addressable locations for placement of molecular probes that can function as sensors, reporters, or enhancers of measurement systems. Embodiments of the present invention may also include a nucleic acid tile structure with addressable locations for placement of molecular probes such that attachment chemistry restricts motion of the probes and permits mixing and matching of probes for multiplexed sensing and detection.

Accordingly, embodiments of the present invention relate to a nucleic acid tile structure of a predetermined size comprising a first template oligonucleotides having a first length comprising a first, a second, a third, a fourth, a fifth, a sixth, and a seventh template domains, wherein the second, the fifth and the sixth template domains are positioned at predetermined locations on the first template oligonucleotides; a second template oligonucleotides having a second length comprising an eighth, a ninth, a tenth, an eleventh, a twelfth, a thirteenth, and a fourteenth template domains, wherein the ninth and the twelfth template domains are positioned at predetermined locations on the second template oligonucleotides, wherein the predetermined size of the nucleic acid tile structure is determined by the first and the second lengths of the first and the second template oligonucleotides; a first staple oligonucleotides comprising a first, a second and a third staple domains, wherein the first staple domain hybridizes to the thirteenth template domain, wherein the second staple domain hybridizes to the first template domain, wherein the third staple domain hybridizes to the seventh template domain, wherein the hybridizing the first, the second and the third staple domains to the first, the seventh and the thirteenth template domains positions the hybridized first staple oligonucleotide to crossover from the first template oligonucleotide to the second template oligonucleotide and from a first portion to a second portion of the first template oligonucleotide; a second staple oligonucleotides comprising a fourth and a fifth staple domains, wherein the fourth staple domain hybridizes to the eighth template domain, wherein the fifth staple domain hybridizes to the fourteenth template domain, wherein the hybridizing the fourth and the fifth staple domains to the eighth and the fourteenth template domains positions the hybridized second staple oligonucleotide to crossover from a first portion to a second portion of the second template oligonucleotide; a third staple oligonucleotides comprising a sixth, a seventh, an eighth and a ninth staple domains, wherein the sixth staple domain hybridizes to the fourth template domain, wherein the seventh staple domain hybridizes to the third template domain, wherein the eighth staple domain hybridizes to the eleventh template domain, wherein the ninth staple domain hybridizes to the tenth template domain, wherein hybridizing the sixth, the seventh, the eighth and the ninth staple oligonucleotides hybridizing to the third, the fourth, the tenth, and the eleventh template domains positions the hybridized third staple oligonucleotide to crossover from the first template oligonucleotide to the second template oligonucleotide and from a third portion to a fourth portion of the first template oligonucleotide and from a third portion to a fourth portion of the second template oligonucleotide; a first probe oligonucleotide comprising nucleotide sequence complementary to the sixth template domain, wherein the first probe oligonucleotide hybridizes to the sixth template domain; a second probe oligonucleotide comprising nucleotide sequence complementary to the fifth template domain, wherein the second probe oligonucleotide hybridizes to the fifth template domain; a third probe oligonucleotide comprising nucleotide sequence complementary to the second template domain, wherein the third probe oligonucleotide hybridizes to the second template domain; a fourth probe oligonucleotide comprising nucleotide sequence complementary to the twelfth template domain, wherein the fourth probe oligonucleotide hybridizes to the twelfth template domain; and a fifth probe oligonucleotide comprising nucleotide sequence complementary to the ninth template domain, wherein the fifth probe oligonucleotide hybridizes to the ninth template domain. In one embodiment, the at least one of the first, the second, the third, the fourth and the fifth probe oligonucleotides further comprises a binding site. More particularly, the binding site can be 5-ethynyl-2′-deoxyuridine.

In one aspect of the present invention, the crossovers of the first, the second, and the third staple oligonucleotide are positioned to fold the first and the second template nucleotide into a predetermined two-dimensional shape, More particularly, the crossovers of the first, the second, and the third staple oligonucleotide can be positioned to fold the first and the second template nucleotide into a rectangular structure having a length of about 10 nm and a width of about 8 nm.

In some embodiments, the nucleic acid tile structure further comprises a plurality of molecular probes, wherein at least one of the plurality of the molecular probes is bound to at least one of the first, the second, the third, the fourth and the fifth probe oligonucleotides. In other embodiments, at least one of the plurality of the molecular probes is bound to at least one of the first, the second, the third, the fourth and the fifth probe oligonucleotides using a covalent bond.

In one embodiment of the present invention, the plurality of the molecular probes is selected from the group consisting of DNA, RNA, polypeptides, lipids, carbohydrates, other organic molecules, inorganic molecules and metallic particles, ferromagnetic particles, and quantum dots. In another embodiment, at least one of the plurality of the molecular probes is a spin label.

In one aspect of the present invention, distances between the first, the second, the third, the fourth and the fifth probe oligonucleotides are from about 3 nm to about 9 nm.

Another embodiment of the present invention relates to a nucleic acid tile structure of a predetermined size comprising a first template oligonucleotides having a first length comprising a first, a second, a third, a fourth, a fifth, a sixth, and a seventh template domains, wherein the second, the fifth and the sixth template domains are positioned at predetermined locations on the first template oligonucleotides; a second template oligonucleotides having a second length comprising an eighth, a ninth, a tenth, an eleventh, a twelfth, a thirteenth, and a fourteenth template domains, wherein the predetermined size of the nucleic acid tile structure is determined by the first and the second lengths of the first and the second template oligonucleotides, wherein the ninth and the twelfth template domains are positioned at predetermined locations on the second template oligonucleotides; a first staple oligonucleotides comprising a first, a second and a third staple domains, wherein the first staple domain hybridizes to the thirteenth template domain, wherein the second staple domain hybridizes to the first template domain, wherein the third staple domain hybridizes to the seventh template domain; a second staple oligonucleotides comprising a fourth and a fifth staple domains, wherein the fourth staple domain hybridizes to the eighth template domain, wherein the fifth staple domain hybridizes to the fourteenth template domain; a third staple oligonucleotides comprising a sixth, a seventh, an eighth and a ninth staple domains, wherein the sixth staple domain hybridizes to the fourth template domain, wherein the seventh staple domain hybridizes to the third template domain, wherein the eighth staple domain hybridizes to the eleventh template domain, wherein the ninth staple domain hybridizes to the tenth template domain; a first probe oligonucleotide hybridized to the sixth template domain, wherein the first probe oligonucleotide further includes a 5-ethynyl-2′-deoxyuridine nucleotide; a second probe oligonucleotide hybridized to the fifth template domain; a third probe oligonucleotide hybridized to the second template domain; a fourth probe oligonucleotide hybridized to the twelfth template domain; a fifth probe oligonucleotide hybridized to the ninth template domain; and a first molecular probe hound to the 5-ethynyl-2′-deoxyuridine nucleotide of the first probe oligonucleotide. In one embodiment, the first molecular probe is a spin label.

In some embodiments, the nucleic acid tile structure further comprises a second, a third, a fourth and a fifth molecular probes bound to the second, the third, the fourth and the fifth probe oligonucleotides.

In one aspect of the present invention, the second, the third, the fourth and the fifth molecular probes is selected from the group consisting of DNA, RNA, polypeptides, lipids, carbohydrates, other organic molecules, inorganic molecules and metallic particles, ferromagnetic particles, and quantum dots.

In another aspect of the present invention, the first, the second, and the third staple oligonucleotide hybridizing to the first and the second template oligonucleotide folds the first and the second template nucleotide into a rectangular shape.

Embodiments of the present invention also relate to a method for making a non-naturally occurring nucleic acid tile structure, said method comprising providing a first template oligonucleotide comprising a first, a second, a third, a fourth, a fifth, a sixth, and a seventh template domains; providing a second template oligonucleotides comprising an eighth, a ninth, a tenth, an eleventh, a twelfth, a thirteenth, and a fourteenth template domains; providing a first staple oligonucleotides comprising a first, a second and a third staple domains; providing a second staple oligonucleotides comprising a fourth and a fifth staple domains; mixing the first template oligonucleotide, the second template oligonucleotide, the first staple oligonucleotide and the second oligonucleotide to form a first mixture; annealing the first mixture to hybridize the first staple domain to the thirteenth template domain, the second staple domain to the first template domain, the third staple domain to the seventh template domain, the fourth staple domain to the eighth template domain, and the fifth staple domain to the fourteenth template domain; providing a third staple oligonucleotides comprising a sixth, a seventh, an eighth and a ninth staple domains; mixing the third staple oligonucleotide with the annealed first mixture to form a second mixture; annealing the second mixture to hybridize the sixth staple domain to the fourth template domain, the seventh staple domain to the third template domain, the eighth staple domain to the eleventh template domain, and the ninth staple domain to the tenth template domain; providing a first, a second, a third, a fourth and a fifth probe oligonucleotides; mixing the first, the second, the third, the fourth and the fifth probe oligonucleotides with the annealed second mixture to form a third mixture; annealing the third mixture to hybridize the first probe oligonucleotide to the sixth template domain, the second probe oligonucleotide to the fifth template domain, the third probe oligonucleotide to the second template domain, the fourth probe oligonucleotide to the twelfth template domain, and the fifth probe oligonucleotide to the ninth template domain; and mixing a plurality of molecular probes to annealed third mixture to bind at least one of the plurality of the molecular probes to the at least one of the first, the second, the third, the fourth and the fifth probe oligonucleotides.

In one embodiment, the first probe oligonucleotide further includes a 5-ethynyl-2′-deoxyuridine nucleotide. In another embodiment, at least one of the plurality of the molecular probes is bound to the 5-ethynyl-2′-deoxyuridine nucleotide of the first probe oligonucleotide. More particularly, at least one of the plurality of the molecular probes bound to the 5-ethynyl-2′-deoxyuridine nucleotide can be a spin label.

In another embodiment, the second, the fifth and the sixth template domains are positioned at predetermined locations on the first template oligonucleotides, and wherein the ninth and the twelfth template domains are positioned at predetermined locations on the second template oligonucleotides.

### BRIEF DESCRIPTION OF DRAWINGS

FIG. 1 illustrates a nucleic acid tile structure in accordance with an embodiment of the present invention.

FIG. 2 illustrates distances between molecular probes in nucleic acid tile structure shown in FIG. 1.

FIG. 3 illustrates a general flow process for generating a nucleic acid tile structure from nucleic acids in accordance with an embodiment of the present invention.

FIG. 4 illustrates a general flow process for spin labeling of probe strands used in an exemplary nucleic acid tile structure in accordance with an embodiment of the present invention.

FIG. 5 illustrates a general flow process for generating a spin-labeled nucleic acid tile structure from nucleic acids in accordance with an embodiment of the present invention.

FIG. 6 illustrates exemplary spectra of a spin-labeled nucleic acid tile structure in accordance with an embodiment of the present invention.

FIG. 7 illustrates alternate nucleic acid tile structures in accordance with embodiments of the present invention.

FIG. 8 illustrates native gel electrophoresis of an alternate nucleic acid tile structure shown in FIG. 7(A) with 1:10 and 1:1 ratios of template strands to other strands.

FIG. 9 illustrates native gel electrophoresis of an alternate nucleic acid tile structure shown in FIG. 7 assembled using varying equivalents of SZ.

### DETAILED DESCRIPTION

As used herein, the singular forms “a”, “an” and “the” include plural referents unless the context clearly dictates otherwise. For example, reference to a “nucleic acid” means one or more nucleic acids.

As used herein, the term “nucleotide” refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

As used herein, the terms “nucleic acid,” “polynucleotide,” and “oligonucleotide” are interchangeable and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, locked nucleic acid, peptide nucleic acid), In general, and unless otherwise specified, an analogue of a particular nucleotide has the same base-pairing specificity, i.e., an analogue of A will base-pair with T. When double-stranded DNA is described, the DNA can be described according to the conformation adopted by the helical DNA, as either A-DNA, B-DNA, or Z-DNA, DNA in the B-form, as described by James Watson and Francis Crick based on Rosalind Franklin's radiographs, is believed to predominate in cells and extends about 3.4 nm per 10 base pairs (bp) of sequence; A-DNA extends about 2.3 nm per 10 bp of sequence, and Z-DNA extends about 3.8 nm per 10 bp of sequence.

As used herein, the terms “oligonucleotide” or a “polynucleotide” are synthetic or isolated nucleic acid polymers including a plurality of nucleotide subunits.

As used herein, the term “domain” refers to a portion of a monomer comprising a sequence.

As used herein, the term “tile” refers to a modular building unit for polynucleotide assembly. In general, a tile has binding sites or binding sections that mediate its interaction with other tiles. In some embodiments, as disclosed herein, the tile is a single-stranded polynucleotide. In some embodiments, the tiles are multi-stranded.

As used herein, each “nucleic acid tile” comprises (a) a structural element (also referred to herein as the polynucleotide “core”) constructed from a plurality of nucleic acid polynucleotides; and (b) 1 or more “sticky ends” per nucleic acid tile attached to the polynucleotide core. A wide range of such polynucleotide cores are possible, including but not limited to 4 arm branch junctions, 3 arm branch junctions, double crossovers, triple crossovers, parallelograms, 8 helix bundles, 6-tube formations, and structures assembled using one or more long strands of nucleic acid that are folded with the help of smaller ‘helper’ strands.

As used herein, the terms “nucleic acid probe”, “probe oligonucleotide”, “probe oligomer” or “probe strand” refer to nucleic acid sequences synthesized as part of one or more polynucleotide structure in a nucleic acid tile that participate in base pairing with other polynucleotide structures within a nucleic acid tile.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Embodiments of the present invention relate, in its broadest sense, to compositions and methods of making nucleic acid tile structures of predetermined, and thus controlled, shape, size and complexity. Embodiments of the present invention is premised, in part, on the unexpected finding that select pluralities of single stranded oligonucleotides can be self-assembled to form nucleic acid tile structures of controlled shape, size, complexity and modification. It was considered surprising, inter alia, that stable nucleic acid structures of various predetermined shapes and controlled sizes could be formed using only a plurality of single stranded oligonucleotides.

Nucleic acid tile structures in accordance with the embodiment of the present invention comprise a plurality of oligonucleotides arranged (via sequence-specific annealing) in a predetermined or known manner. As a result, the position of each oligonucleotide in the structure is known. In this way, the structure may be modified, for example through attachment of moieties, at predetermined positions. This may be accomplished by using a modified oligonucleotide as a starting material or by modifying a particular oligonucleotide after the structure is formed. Therefore, knowing the position of each of the starting oligonucleotides in the resultant structure provides addressability to the structure.

Nucleic acid tile structures in accordance with the embodiment of the present invention may be made, in some instances, through a process of self-assembly of single stranded oligonucleotides. In these self-assembly methods, the single stranded oligonucleotides are combined in a single vessel and allowed to anneal to each other, based on sequence complementarity. In some instances, this annealing process involves placing the oligonucleotides at an elevated temperature and then reducing the temperature gradually in order to favor sequence-specific binding. As used herein, the term “self-assembly” refers to the ability of oligonucleotides (and in some instances nucleic acid structures) to anneal to each other, in a sequence-specific manner, in a predicted manner and without external control (e.g., by sequential addition of oligonucleotides or nucleic acid structures).

Embodiments of the present invention therefore provides, inter alia, compositions comprising the single stranded oligonucleotides of the invention, methods of making nucleic acid tile structures of various predetermined or known size, shape, complexity and modification, nucleic acid structures of various predetermined or known size, shape, complexity and modification, pluralities of nucleic acid structures wherein such pluralities may be substantially monodisperse with respect to size, shape, complexity and modification, composition structures comprising two or more nucleic acid structures, and methods of making such composite structures. Embodiments of the present invention also provides methods of using the nucleic acid tile structures and the composite structures of the invention. These aspects and embodiments of the invention will be described in greater detail herein.

Embodiments of the present invention described herein relate to compositions and methods pertaining to nucleic acid tile structures that are also capable of providing addressable locations for placement of molecular probes.

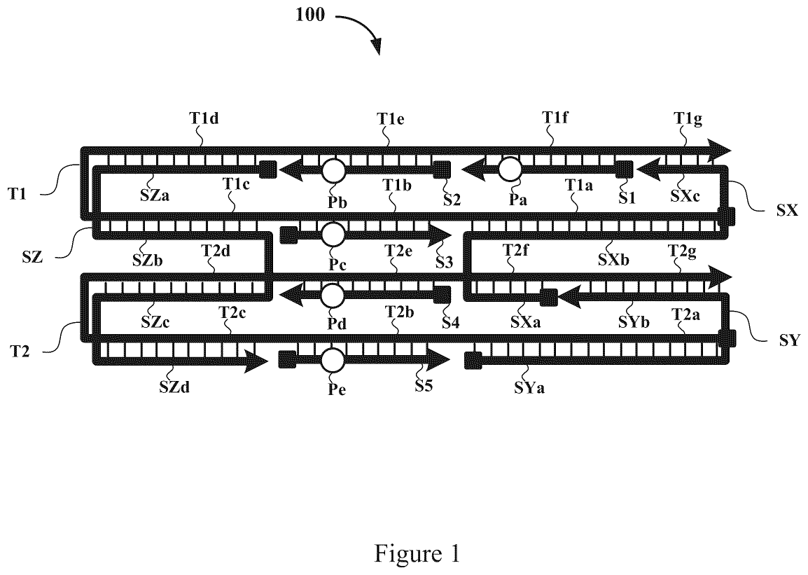

Referring now to the drawings, and more particularly, to FIG. 1, there is shown a schematic depiction of a structure of a nucleic acid tile, generally designated 100 and schematically showing an embodiment of the present invention, for providing addressable locations for placement of molecular probes. Nucleic acid tile structure 100 in accordance with embodiments of the present invention include a plurality of template oligomers (also referred to as template strands) T1 and T2, a plurality of staple oligomers (also referred to as staple strands) SX, SY and SZ, a plurality of probe oligomers (also referred to as probe strands) S1-S5, and a plurality of molecular probes Pa-e assembled in a set order and into a prescribed two-dimensional structure.

Template oligomers T1 and T2 guide the assembly of nucleic acid tile structures 100 and provide the geometric parameters and physical dimensions to nucleic acid tile structures 100. Nucleic acid tile structure 100 in accordance with embodiments of the present invention can be formed using one or more template oligomers. In some embodiments of the present invention, nucleic acid tile structure 100 is formed using a single-stranded template oligomer. In other embodiments of the present invention, nucleic acid tile structure 100 is formed using multi-stranded template oligomers. In one embodiment of the present invention, nucleic acid tile structure 100 is formed using two template oligomers. Template oligomers T1 and T2 can include natural nucleic acids or non-natural nucleic acids, or can include a combination of natural and non-natural nucleic acids. In some embodiments, one or more known nucleic acid sequences are used as a default template sequence. In one embodiment, a default template sequence for each of T1 and T2 is a sequence or a subset of a sequence corresponding to those shown in Table 1.

TABLE 1

| Name | Sequence |
|---|---|
| T1 | AGC CTC GTC TGT TCT CCC CGC TAA CGA ACT CAA ACC CGG GGC CCG |
| | ACG CGA CAT ATC AGC TAA GAG TAG GCC GGG GAA AGA CAA GCA GAC |
| | GT (SEQ ID NO: 1) |
| T2 | AGC CTC GTC TGT TCT CCC CTA ATC AGC TTC AAC GAG CCG TAC AGG |
| | TGG CAC CTC AGG AGG GGC CCA CAG GGA GGG GAA AGA CAA GCA G?C |
| | GT (SEQ ID NO: 2) |
| S1 | TGC TTG TCT TTC CCC T (SEQ ID NO: 3) |
| S2 | CCC TGT GGG CCC CTC C (SEQ ID NO: 4) |
| S3 | TCG TTG AAG CTG ATT A (SEQ ID NO: 5) |
| S4 | CGG CCT ACT CTT AGC T (SEQ ID NO: 6) |
| S5 | GGG TTT GAG TTC GTT A (SEQ ID NO: 7) |
| SX | CTT TCG CGG GGA GAA CAG ACG AGG CTT AAC GTC (SEQ ID NO: 8) |
| SY | GCG GGG AGA ACA GAC GAG GCT TAA CGT CTG TTG T (SEQ ID NO: 9) |
| SZ | TGA GGT GCC ACC TGT ACG GCG ATA TGT CGC GTC GGG CCC C (SEQ ID |
| | NO: 10) |

Each of template oligomers T1 and T2, represented as a square U-shaped structure in FIG. 1, comprises a plurality of concatenated domains. Generally, every domain of a template oligomer binds to another domain in another oligonucleotide structure. A domain region includes a nucleic acid sequence configured to hybridize to a complementary nucleic acid sequence of another oligonucleotide. In an embodiment of the present invention, as shown in FIG. 1, template oligomers T1 and T2 can comprise fourteen concatenated domains T1a-g and T2a-g. In one embodiment of the present invention, each of template oligomers T1 and T2 include seven domains, as shown in FIG. 1. Each domain is characterized by its sequence and its length, as discussed herein. In one embodiment of the present invention, domains Tia-n present in template oligomer T1 and domains T2a-g present in template oligomer T2, when hybridized to a corresponding oligonucleotide, form a rectangular structure having a length of about 10 nm and width of about 8 nm.

Domains Tia-g and T2a-g in template oligomers T1 and T2 of nucleic acid tile structure 100 in accordance with embodiments of the present invention, as described herein, can be of various lengths. For example, T1 and T2 can both be about 92 nucleotides in length, or domains T1a-g can be about 20, 16, 10, 10, 16, 16, and 4 nucleotides in length and T2a-g can be about 20, 16, 10, 10, 16, 4, and 16 nucleotides in length. In some embodiments, all domains T1a-g and T2a-g in template oligomers T1 and T2 are of the same length. In another embodiment, domains T1a-g and T2a-g in template oligomers T1 and T2 are in linked form and are about 20, 16, 10, 10, 16, 4, 16, 20, 16, 10, 10, 16, 16, and 4 nucleotides in length. In one embodiment, domains T1a-g and T2a-g in template oligomers T1 and T2 are each 24 nucleotides in length. In another embodiment, domains T1a-g and T2a-g are each 42 nucleotides in length. In some embodiments, domains T1a-g are each 32 nucleotides in length.

Staple oligomers SX, SY and SZ include short single strands of nucleic acids that direct the folding or bending of template oligomers T1 and T2 into desired shapes and sizes, and structures therefrom. Nucleic acid tile structure 100 in accordance with embodiments of the present invention can be formed using one or more staple oligomers. Staple oligomers SX, SY and SZ include domains that bind to target domains of template oligomers T1 and T2. In some embodiments, a default sequence for each of SX, SY and SZ is a sequence or a subset of a sequence corresponding to those shown in Table 1. In an embodiment of the present invention, as shown in FIG. 1, staple oligomer SX can include three concatenated domains SXa-c, staple oligomer SY can include two concatenated domains SYa-b, and staple oligomer SZ can include four concatenated domains SZa-d. As further illustrated in FIG. 1, staple oligomers SX, SY and SZ may be arranged such that nucleic acid tile structure 100 includes oligonucleotides crossovers, half crossovers, or a combination thereof. Staple oligomers SX-SZ may also be arranged such that oligonucleotides crossovers and/or half crossovers occur at different distances including but not limited to every two domains or every four domains, and the like.

As illustrated in FIG. 1, staple oligomer SX binds to template oligomer T1 through hybridization of nucleic acid sequence of domain SXb with corresponding complementary nucleic acid sequence of domain Tia and through hybridization of nucleic acid sequence of domain SXc with corresponding complementary nucleic acid sequence of domain T1g. As further illustrated FIG. 1, staple oligomer SX may be arranged such that at least one portion of staple oligomer SX includes oligonucleotides that crossover to template oligomer T2 such that staple oligomer SX also binds to template oligomer T2 through hybridization of nucleic acid sequence of domain SXa with corresponding complementary nucleic acid sequence of domain T2f. Domains T1a, T1g and T2f are positioned to constrain crossovers of hybridized staple oligomer SX from template oligomer T1 to template oligomer T2 and between portions of template oligomer T1, as shown in FIG. 1.

Staple oligomer SY binds to template oligomer T2 through hybridization of nucleic acid sequence of domain SYa with corresponding complementary nucleic acid sequence of domain T2a and through hybridization of nucleic acid sequence of domain SYb with corresponding complementary nucleic acid sequence of domain T2g. Domains T2a and T2g are positioned to constrain crossovers of hybridized staple oligomer SY between portions of template oligomers T2, as shown in FIG. 1.

Staple oligomer SZ binds to template oligomer T1 through hybridization of nucleic acid sequence of domain SZa with corresponding complementary nucleic acid sequence of domain T1d and through hybridization of nucleic acid sequence of domain SZb with corresponding complementary nucleic acid sequence of domain T1c. As further illustrated FIG. 1, staple oligonucleotide SZ may be arranged such that at least one portion of staple oligomer SZ includes oligonucleotides that crossover to template oligomer T2 such that staple oligomer SZ also binds to template oligomer T2 through hybridization of nucleic acid sequence of domain SZc with corresponding complementary nucleic acid sequence of domain T2d and through hybridization of nucleic acid sequence of domain SZd with corresponding complementary nucleic acid sequence of domain T2c. Domains T1c, T1d, T2c and T2d are positioned to constrain crossovers of hybridized staple oligomer SZ from template oligomer T1 to template oligomer T2 and between portions of template oligomers T1 and T2.

Embodiments of the present invention contemplate various binding arrangements for oligonucleotides within a nucleic acid tile structure. In some instances, however, certain domains in a nucleic acid tile structure may not bind to another domain in the structure. As an example, in some instances, domains having a poly T domain are present in the structure, preferably at borders and in configurations that result in the poly T domains being single stranded. As another example, domains may be used as handles for annealing to other structures or to other moieties.

Probe oligomers S1-S5 include nucleic acid sequences that bind to predetermined parts of nucleic acid tile structure 100 that do not participate in binding with staple oligomers SX, SY and SL within nucleic acid tile structure 100. Thus, probe oligomers S1a-e include nucleic acid sequences that are available for binding with template oligomers T1 and T2. Probe oligomer S1 binds to template oligomer T1 through hybridization of nucleic acid sequence of probe oligomer S1 with corresponding complementary nucleic acid sequence of domain T1f, probe oligomer 52 binds to template oligomer T1 through hybridization of nucleic acid sequence of probe oligomer S2 with corresponding complementary nucleic acid sequence of domain T1c, and probe oligomer S3 binds to template oligomer T1 through hybridization of nucleic acid sequence of probe oligomer S3 with corresponding complementary nucleic acid sequence of domain T1b. As further illustrated FIG. 1, probe oligomer 54 binds to template oligomer T2 through hybridization of nucleic acid sequence of probe oligomer S4 with corresponding complementary nucleic acid sequence of domain T2e, and probe oligomer S5 binds to template oligomer T2 through hybridization of nucleic acid sequence of probe oligomer S5 with corresponding complementary nucleic acid sequence of domain T2b. In one embodiment, a default sequence for each of S1-55 is a sequence or a subset of a sequence corresponding to those shown in Table 1.

Probe oligomers S1-S5 are capable of binding with molecular probes Pa-e either by direct bind or by indirect binding, or a combination thereof. In one embodiment of the present invention, molecular probe Pa binds to probe oligomer S1, molecular probe Pb binds to probe oligomer S2, molecular probe Pc binds to probe oligomer S3, molecular probe Pd binds to probe oligomer S4, and molecular probe Pe binds to probe oligomer S5. In one embodiment of the present invention, only one of molecular probes Pa-e is bound to one of probe oligomers S1-S5. In other embodiments of the present invention, one or more of molecular probes Pa-e is bound to one of probe oligomers S1-S5. In some embodiments of the present invention, each of molecular probes Pa-e bound to probe oligomers S1-S5 is similar. In other embodiments of the present invention, each of molecular probes Pa-e bound to probe oligomers S1-S5 are a combination of different types of molecular probes.

As used herein, “direct binding” means that molecular probes Pa-b bind directly to probe oligomers S1-S5. Such binding can be of any type, including base pairing with nucleic acids, or other interactions. Exemplary molecular probes Pa-b for direct interaction include nucleic acids (DNA and RNA whether single stranded or double stranded; DNAzymes, aptameric sensors, signaling aptamers), polypeptides, lipids, carbohydrates, other organic molecules, inorganic molecules (including but not limited to insulators, conductors, semi-conductors, magnetic particles, metallic particles, optical sensors, etc.), ferromagnetic particles, quantum dots, and any other type of molecule to which probe oligomers S1-S5 are capable of binding, and in combination thereof. As used herein, “indirect binding” means that molecular probes Pa-b binds to probe oligomers S1-S5 through some intermediate molecule. Non-limiting examples of indirect binding would include chemical conjugation approaches that facilitate the formation of certain DNA-peptides, DNA-PNA, and PNA-Peptides, chimeric molecules, as well as other molecular biology approaches like ribosome display and DNA display. Those skilled in the art will recognize, based on the teachings herein, that any other molecules can be indirectly bound to the nucleic acid probe of the invention, including but not limited to nucleic acids (DNA and RNA whether single stranded or double stranded), lipids, carbohydrates, other organic molecules, inorganic molecules and metallic particles, ferromagnetic particles, and quantum dots. Conditions for binding molecular probes Pa-e to probe oligomers will depend on the nature of the probe and the target, but can be determined by those skilled in the art, based on the teachings herein.

The chemistry used to bind molecular probes Pa-e covalently to probe oligomers S1-S5 is determined by the type of nucleic acid modification that exists in each probe strand (e.g. modifications that support click chemistry, disulfide bond formation, amide bond formation, etc). In some embodiments of the present invention, probe oligomers S1-S5 include a modified nucleotide capable of binding to molecular probes Pa-e. In an exemplary embodiment of the present invention, the modified nucleotide capable of binding to molecular probes Pa-e is 5-ethynyl-2′-deoxyuridine (dU*). Table 2 provides exemplary sequences with dU* binding sites for probe oligomers S1-S5 in an arrangement as shown in FIG. 1.

TABLE 2

| Staple | Sequence |
|---|---|
| S1 | TGC TTG TCT TU*C CCC T (SEQ ID NO: 11) |
| S2 | CCC TGU* GGG CCC CTC C (SEQ ID NO: 12) |
| S3 | TCG TTG AAG CU*G ATT A (SEQ ID NO: 13) |
| S4 | CGG CCU* ACT CTT AGC T (SEQ ID NO: 14) |
| S5 | GGG TTT GAG TU*C GTT A (SEQ ID NO: 15) |

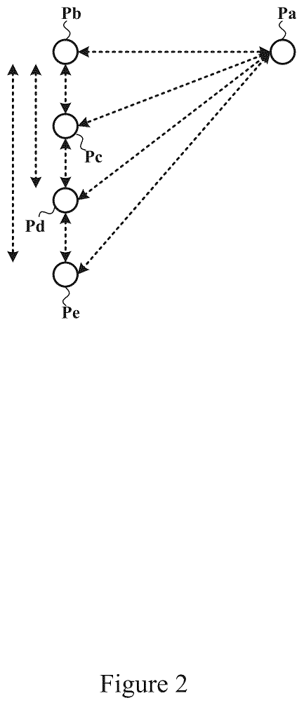

Varying which probe strands are covalently labeled with specific molecular probes allows synthesis of nucleic acid tile structures with control over predetermined inter-probe distances, as shown in FIG. 2. In one embodiment, inter-probe distances between five probe oligomers S1-S5, as shown in FIG. 2, can range from about 3 nm to about 9 nm. Table 3 provides exemplary inter-probe distances between probe oligomers S1-S5 in an arrangement as shown in FIGS. 1 and 2.

TABLE 3

| | Probe Strand | | | | |
|---|---|---|---|---|---|
| | | S1 | S2 | S3 | S4 |
| Probe | S2 | 36.7 ± 1.1 Å | | | |
| Strand | S3 | 44.2 ± 2.5 Å | 41.6 ± 1.6 Å | | |
| | S4 | 59.2 ± 1.7 Å | 48.1 ± 0.7 Å | 33.8 ± 1.0 Å | |
| | S5 | 82.6 ± 2.0 Å | 84.8 ± 1.2 Å | 49.1 ± 0.5 Å | 44.0 ± 1.8 Å |

Molecular probes Pa-b can be detected using electrical, magnetic, or optical methods. In one embodiment, each of molecular probes Pa-e is a spin label capable of being used in electron paramagnetic or nuclear magnetic resonance. In another embodiment, each of molecular probes Pa-b is a contrast agent capable of being used in nuclear magnetic resonance or magnetic resonance imaging. In some embodiment, each of molecular probes Pa-b is a fluorescent molecule capable of being used in optical microscopy.

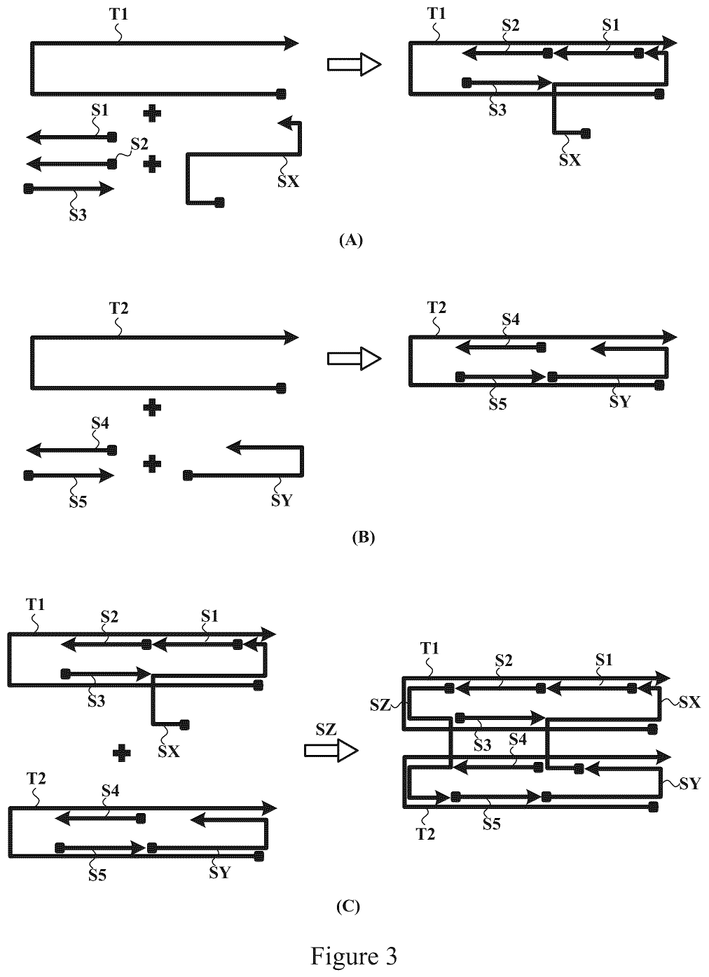

Nucleic acid tile structure 100 in accordance with embodiments of the present invention can be assembled by mixing template oligonucleotides T1 and T2 with staple oligonucleotides SX and SY, usually in a large molar excess of staple oligonucleotides SX and SY, followed by annealing the mixture to hybridize staple domains SXa to template domain T2f, hybridize staple domains SXb to template domain T1a, hybridize staple domains SXe to template domain T1g, hybridize staple domains SYa to template domain T2a, and hybridize staple domains SYb to template domain T2g, as shown schematically in FIGS. 3(A) and 3(B). The annealed mixture is mixed with staple oligonucleotide SZ and further annealed to hybridize staple domains SZa to template domain T1d, hybridize staple domains SZb to template domain T1c, hybridize staple domains SZc to template domain T2d, and hybridize staple domains SZd to template domain T2c, as further shown schematically in FIG. 3(C). Staple oligonucleotides SX, SY and SZ hybridized to template oligonucleotides T1 and T2 fold template oligonucleotides T1 and T2 into a predetermined shape having a predetermined dimensions. In one embodiment, probe oligomers S1-S5 and staple oligonucleotides SX, SY and SZ are mixed with the template oligonucleotides T1 and T2 hybridized with and further annealed to hybridize probe oligonucleotide S1 to template domain T1f, hybridize probe oligonucleotide S2 to template domain T1e, hybridize probe oligonucleotide S3 to template domain T1 b, hybridize probe oligonucleotide S4 to template domain T2e, and hybridize probe oligonucleotide S5 to template domain T2b to form nucleic acid tile structure 100. In another embodiment of the present invention, probe oligomers S1-S5 with molecular probes Pa-e attached to probe oligomers S1-S5 are mixed with staple oligonucleotides SX, SY and SZ and the template oligonucleotides T1 and T2 and annealed to hybridize probe oligonucleotide S1 to template domain T1f, hybridize probe oligonucleotide S2 to template domain T1e, hybridize probe oligonucleotide S3 to template domain T1b, hybridize probe oligonucleotide S4 to template domain T2e, and hybridize probe oligonucleotide S5 to template domain T2b to form nucleic acid tile structure 100.

Covalently attaching a molecular probe to probe oligomers S1-S5 before or after nucleic acid tile structure 100 assembly depends on multiple factors, which can include, but are not limited to, the heat stability of the molecular probe, the covalent attachment chemistry to be employed, and the number of molecular probes to be incorporated in the final structure. To generate nucleic acid tile structure 100 assemblies with molecular probes that are heat sensitive, the template, staple, and probe strands (lacking attached, heat-sensitive molecular probe) are combined and annealed. Post-annealing, approximately one mole equivalent of probe strand containing the heat-sensitive, covalently attached molecular probe is added. The resulting mixture is incubated for 1 h at room temperature prior to purification using centrifugal filtration devices with a nominal molecular mass cutoff of 100 kDa. Assembly of nucleic acid tile structure 100 can be assessed using standard non-denaturing gel electrophoresis.

Reference now to the specific examples which follow will provide a clearer understanding of systems in accordance with embodiments of the present invention. The examples should not be construed as a limitation upon the scope of the present invention.

### Example 1. Preparation of a Nucleic Acid Tile

As a specific example of preparation of a nucleic acid tile structure 100 in accordance with embodiments of the present invention, aliquots of stock solutions of template strands T1 and T2, staple strands SX, SY and SZ, and probe strands S1-S5 are combined in tris(hydroxymethyl)aminomethane (Tris, 25 mmol L⁻¹)-acetic acid buffer solution (pH 7.5) containing 2 mmol magnesium acetate in thin-walled polymerase chain reaction (PCR) sample tubes. Probe strands S1-S5 can be unlabeled or covalently labeled with molecular probe prior to assembly of the tile. The volumes of stock solutions of template strands T1 and T2, staple strands SX, SY and SZ, and probe strands S1-S5 to be combined are chosen to produce the desired stoichiometries and concentrations with final volumes ranging from 50 uL to 120 uL. The starting template strands T1 and T2 concentrations are 100 nmol L⁻¹ for non-denaturing gel electrophoresis analysis. The concentration of staple strand SZ is equal to that of the template strands T1 and T2; all other staple and probe strands are present in 10-fold excess over the template strands T1 and T2. The resulting solution containing template, staple and probe strands in buffer is annealed in a PCR thermal cycler over 12 h (95 deg. C. to 25 deg. C. with a rate of 2 deg. C./h and a final hold at 4 deg. C.). Fully assembled tile is isolated using centrifugal filtration devices with a nominal molecular mass cutoff of 100 kDa. Assembly of the nucleic acid tile structure was assessed using standard non-denaturing gel electrophoresis.

### Example 2. A DNA Tile Electron Spin Breadboard

Selective and covalent attachment of stable paramagnetic molecules (spin labels) on biomolecular systems is typically used for structural and dynamics measurements based on electron paramagnetic resonance (EPR) spectroscopy. Double electron-electron resonance (DEER) determines the magnitude of the dipole-dipole interaction and relates that quantity to an inter-spin distance, which provides structural information. Such measurements are carried out by measuring pairwise dipolar interactions on a biomolecular library in which each sample contains a pair of spin labels. Conducting DEER measurements on a single sample containing three or more spin labels, as compared to several samples each containing a pair of labels, could reduce sample preparation time and increase measurement throughput. The “three (or more) labels” approach has been demonstrated for several model systems. Molecular systems generated via multi-step organic synthesis procedures provide a rigid framework for organizing and systematically increasing the number of unpaired electrons and have been important in development of theoretical treatments to deconvolve the increased number of inter-spin contributions. These stable systems can serve as distance measurement “rulers” and as checks/monitors of instrumentation performance over time. A disadvantage for most laboratories is the complex chemical synthesis often required to obtain such compounds. Biomolecular systems with three or more unpaired electron spins also are available; examples are oligomers with a spin label on each monomer (GroEL, amyloids) and three spin labels on a single protein, including systems with three different spin label types (Gd, Mn, NO). The steps required to engineer a biomolecular system for attachment of three (or more) identical spin labels are the same as for a two-spin label construct, making routine production simpler than for the chemically synthesized examples given above. To incorporate non-identical spin labels, orthogonal attachment chemistries are required, the exception being biopolymer heterodimers, heterotrimers, etc. in which each component can be labeled independently prior to complex formation.

For all the systems above, systematically increasing the number of spins while retaining the ability to change the position of spins on the scaffold may not possible. To provide this increased versatility, an electron spin breadboard has been fabricated by harnessing the versatility and performance of DNA nanostructures in organizing objects at the nanoscale. A DNA tile that is capable of placing up to 5 spin labels at defined positions resulting in unique distances has been developed. Use of a DNA tile reduces costs and increases final concentrations compared to use of a DNA origami-based system. The DNA tile has dimensions of approximately 10 nm×8 nm, roughly the outer limits of inter-spin distance measurements via pulsed dipolar spectroscopies. The sequence-specificity of DNA makes incorporation of different spin label types at defined positions in the structure straightforward when an azide-derivative of the spin label is available. The following provides the design, assembly, and EPR spectroscopy of a DNA tile that has been singly-spin labeled in two different positions or doubly-spin labeled. The DNA tile assembles that spin-labeled 16-mer deoxyoligonucleotides can be incorporated into the structure, and that purification of the fully assembled, spin-labeled constructs is simple.

It is contemplated that the DNA tile can be combined with other DNA nanostructures to create multifunctional materials.

Tile design: DNA structure designing tool (Cadnano and CanDo) were used to evaluate candidate structures and molecular dynamics (MD) simulations were performed to determine inter-spin distances.

Materials: Templates and staples with or without 5-ethynyl-2′-deoxyuridine (dU*) were purchased from a commercial supplier of custom deoxyoligonucleotides. Deoxyoligonucleotide sequences for all DNA tile designs are provided in Table 1. Concentrations of oligonucleotide stock solutions in water were determined using absorption spectroscopy at 260 nm (A260) and the vendor-provided extinction coefficients. No correction was applied to the extinction coefficients for the oligonucleotides containing 5-ethynyl-deoxyuridine. Samples were prepared in tris(hydroxymethyl)aminomethane (Tris, 25 mmol L⁻¹)-acetic acid buffer (0.5×TA, pH 7.5) containing 2 mmol L⁻¹ magnesium acetate for assembly/annealing. Gels were poured and run with 0.5×TA buffer. A 19:1 acrylamide:bis-acrylamide solution was used for the gels.

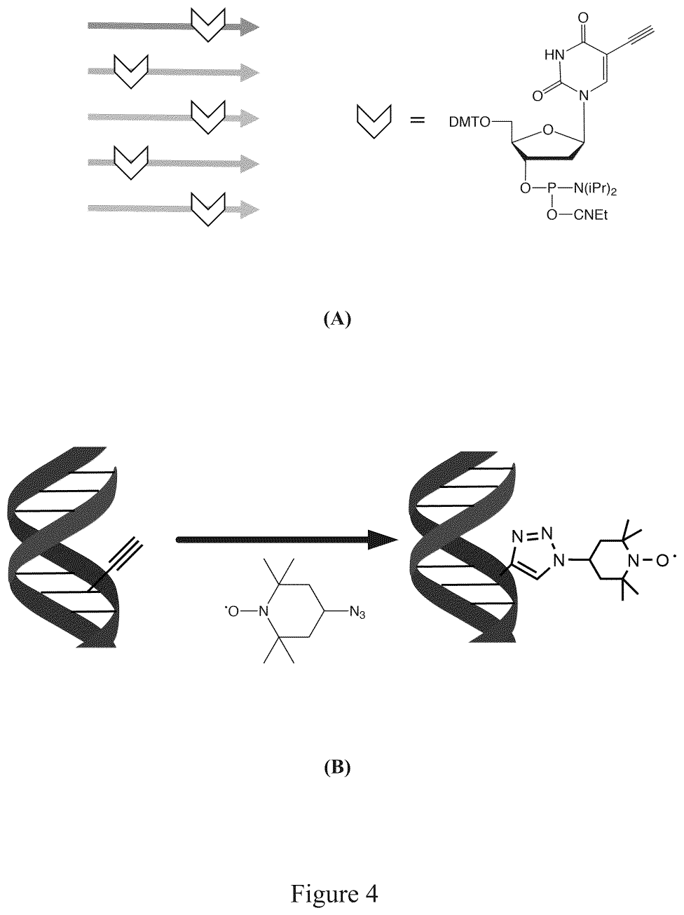

Spin labeling of dU*-containing staples 2 & 4: Spin label is attached to probe strands containing 5-ethynyl-dU via click chemistry, as shown in FIGS. 4(A) and 4(B). Amber glass vials with septa were used as reaction vessels. Alkyne-containing staples 2 & 4 were dissolved in water to a concentration of 1 mmol A solution of 0.1 mol L⁻¹ sodium ascorbate was prepared in water immediately prior to carrying out the spin-labeling reaction. Two reactant solutions were prepared. Solution 1 consisted of 200 mmol L⁻¹ (2,2,6,6-Tetramethylpiperidin-1-yl)oxyl-azide (TEMPO-N₃) in a mixture of dimethylsuifoxide (DMSO) and ethanol (EtOH) (3:1 vol/vol); this TEMPO-N₃ solution was stored at −80 deg. C. Solution 2 contained 0.05 mol 1:1 copper sulfate with 0.1 mol L⁻¹ tris-hydroxypropyltriazolylmethylamine (THPTA) in a mixture of DMSO and EtOH (3:1 vol/vol); this Cu/THPTA solution was stored at 4 deg. C. The Cu/THPTA solution was prepared by mixing equal volumes of a 0.1 mol L⁻¹ copper sulfate solution in water with a 0.2 mol L⁻¹ THPTA solution in DMSO/EtOH (3:1 v/v).

To each septum-capped glass vial was added 50 of 1 mmol L⁻¹ dU*-containing staple in water and 12 uL of Solution 1 (200 mmol L⁻¹ TEMPO-N₃ in DMSO/EtOH). The resulting solution was degassed for several minutes with a gentle argon flow through the septum using a needle for argon input and a vent needle. Under argon, 30 μL of Solution 2 (0.05 mol L⁻¹ copper sulfate/0.1 mol L⁻¹ THPTA in DMSO/EtOH) was added to the vial via gas-tight syringe. Subsequently, 30 uL of freshly prepared 0.1 mol L⁻¹ sodium ascorbate was added, also via gas-tight syringe. The solution in each vial was degassed for an additional 5 min under a gentle argon purge. The vials were removed from the argon purge and shaken gently overnight at 4 deg. C. The reactions were worked up on gel filtration spin columns (solid phase protein fractionation range of 1 kDa to 5 kDa; dextran fractionation range of 100 Da to 5 kDa) followed by concentration in centrifugal filtration devices (nominal molecular weight cutoff of 3 kDa). The gel filtration column eluent was combined with 250 μL of 0.5×TA buffer prior to being added to the centrifugal filtration device. The sample in the centrifugal filtration device was concentrated for 20 min at 14 000×g at 4 deg. C. After the initial concentration step, 300 μL of 0.5×TA buffer was added to the sample and the sample was concentrated again for 20 min at 14 000×g; this step was repeated. The final concentrates of spin-labeled 16-mer staple strand were collected and stored at −80 deg, C until use.

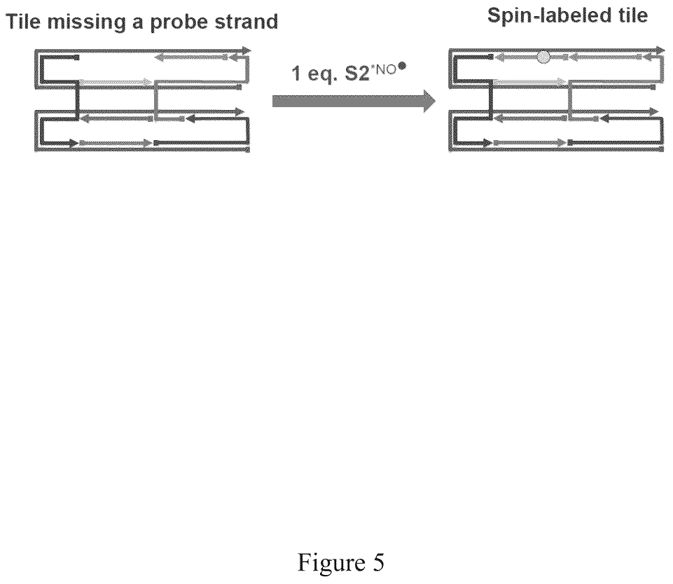

Tile preparation: Template concentrations were approximately 100 nmol L⁻¹ in all samples used for gel electrophoresis. Stoichiometries of the other DNA strands in the samples varied depending on the experiment and are given in the figure legends. Template concentrations were 40 mmol L⁻¹ for assembly of spin-labeled tiles for EPR spectroscopy measurements; stoichiometries of other DNA strands are provided in Table 4. Typically, the concentration of SZ was equal to that of the template strands, see Table 4. Aliquots of DNA strand, magnesium acetate, and TA buffer stock solutions were combined in thin-walled polymerase chain reaction (PCR) tubes to produce the desired stoichiometries and concentrations for a particular experiment. Sample volumes ranged from 50 μL to 120 μL depending on the experiment. Samples were annealed in a PCR thermal cycler over 12 h (95 deg. C. to 25 deg. C. and a final hold at 4 deg. C.). For tile assemblies containing spin-labeled staples, tiles were annealed without addition of spin-labeled staple strand(s). Post-annealing, approximately 1 equivalent of spin-labeled staple was incubated with the tile sample for 1 h prior to gel electrophoresis and/or purification of the final structure, as shown in FIG. 5. Fully assembled tile was isolated by centrifugal filtration devices with a nominal cutoff of 100 kDa.

TABLE 4

| Reagent: | Concentration relative to T1 |
|---|---|
| T1 | 1× |
| T2 | 1× |
| SX | 10× |
| SY | 10× |
| SZ | (1 to 2)× |
| S1-S5 | 10× |

Non-denaturing polyacrylamide gel electrophoresis (PAGE): 16 cm×0.5 mm 6% PAGE gels were pre-run at 100 V to 150 V for 15 mins at 4 deg. C. prior to loading samples. Samples (10 uL) for gel-loading were prepared by combining 6 uL of DNA-containing sample with 4 uL of native dye (in glycerol/0.5×TA buffer 1:1 vol/vol). Gels were run for 6 h to 7 h at 100 V to 150 V. Gels were stained with SYBR green in buffer for at least 30 mins prior to imaging on a UV-transilluminator.

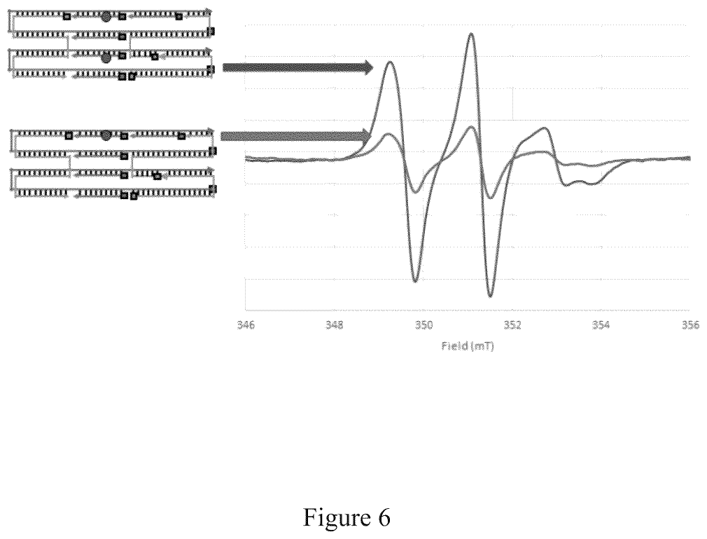

Electron Paramagnetic Resonance (EPR) Spectroscopy: Continuous Wave EPR spectra of nitroxide-labeled DNA (FIG. 6) were collected at room temperature on a commercial spectrometer operating at 9 GHz. FIG. 6 illustrates spectra characterizing a nucleic acid tile structure in accordance with an embodiment of the present invention including one or two spin label probes. Spectral collection conditions are indicated in the figure legends.

Pulsed double electron-electron resonance (DEER) spectroscopy measurements were carried out at temperatures of 40 K to 80 K on a spectrometer equipped with a resonator compatible with pulsed electron paramagnetic resonance measurements. Samples for DEER were loaded into quartz sample tubes. The spin label concentration in the samples for DEER were in the range of about 50 μmol L⁻¹ to about 500 μmol L⁻¹ based on spin quantitation carried out by continuous wave electron paramagnetic resonance measurements. The pulse sequences were selected to acquire DEER data with adequate signal to noise for analysis.

Results: Various combinations of labeling positions & types of labels were contemplated: multiple short strands with functional group for covalent attachment of labels; unique sequences of each of these short oligos to ensure label is directed to intended location on tile.

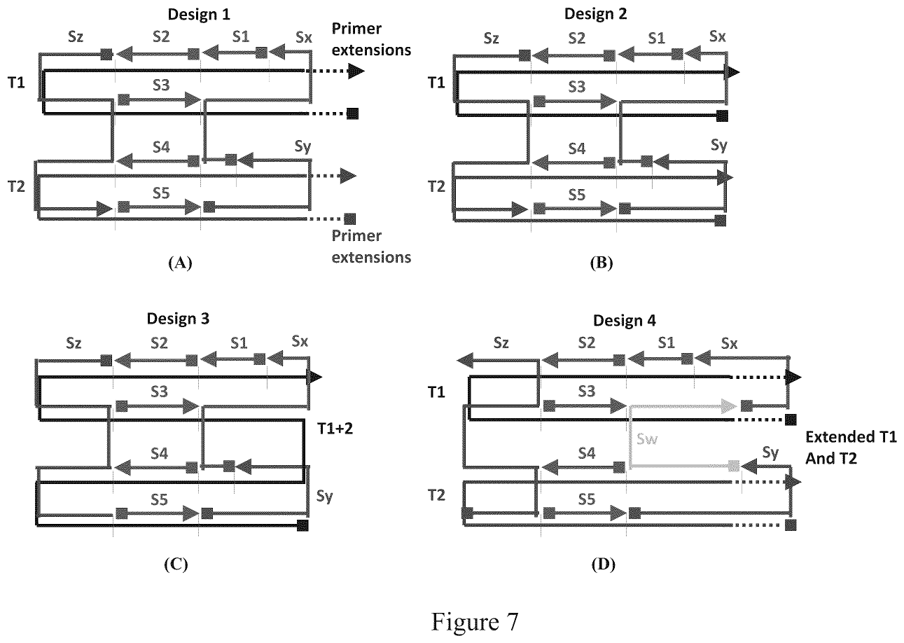

Various DNA tile designs have been contemplated, as shown in FIG. 7. In DNA tile design #1, as shown in FIG. 7(A), PCR sequences at ends of templates enabled sequence duplication. DNA tile design #2 removed sequence duplication and retained types and base numbers the same, as shown in FIG. 7(B). DNA tile design #3 linked. T1 and T2, and DNA tile design #4 adjusted crossover positions, as shown in FIGS. 7(C) and 7(D). Table 5 provides exemplary sequences for T1-2, T2-2, SX-2 and SY-2 in design #2. SZ-2 is unchanged in design #2.

TABLE 5

| | |
|---|---|
| T1-2 | AGC CTC GTC TGT TCT CCC CGC TAA CGA ACT CAA ACC CGG GGC CCG ACG |
| | CGA CAT ATC AGC TAA GAG TAG GCC GGG GAA AGC TAG GAC CAA AG (SEQ ID |
| | NO: 16) |
| T2-2 | GGT CAC TTA GCG AAC TCT GTA ATC AGC TTC AAC GAG CCG TAC AGG TGG |
| | CAC CTC AGG AGG GGC CCA CAG GGA GGG GAA AGA CAA GCA GTG AC (SEQ |
| | ID NO: 17) |
| SX-2 | CTT TCC CC AGA GTT CGC TAA GTG ACCT AGT CAC (SEQ ID NO: 18) |
| SY-2 | GCG GGG AGA ACA GAC GAG GCT TAC TTT GGT CCT AG (SEQ ID NO: 19) |

Determining optimal assembly conditions for the DNA tile required variation of many parameters and conditions: Mg²⁺ concentration, strand stoichiometries, annealing procedure, gel resolving power (bis-acrylamide content), and purification protocols.

The first parameter tested was the Mg²⁺ concentration. Two concentrations were tested: 12 mmol/L and 2 mmol/L. The higher concentration was chosen because DNA origami systems are usually assembled in the presence of >10 mmol L⁻¹ Mg²⁺. The lower concentration was selected because the DNA tile contains many fewer DNA strands than DNA origami and, therefore, may require less screening of backbone charge by divalent cations, Both concentrations supported tile assembly; the lower Mg²⁺ concentration was selected for subsequent experiments. Initially, Mg²⁺ was included in the DNA tile assembly/annealing and gel electrophoresis steps, but some DNA did not migrate out of the wells when Mg²⁺ was present in the gel electrophoresis buffer. Removal of Mg²⁺ from the gel electrophoresis buffer corrected this problem.

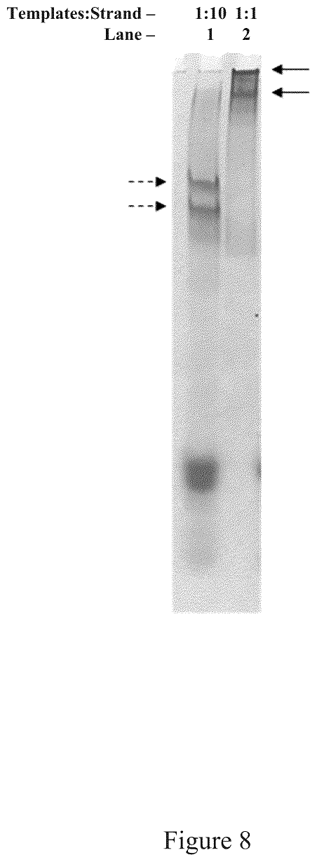

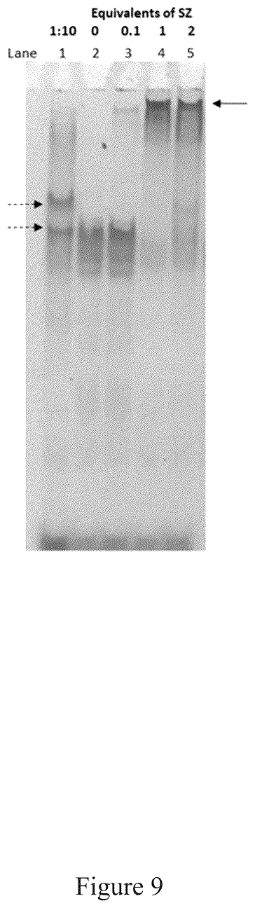

Performing stepwise assembly experiments and testing staple stoichiometries were important because conditions for generating the highest tile yields typically do not transfer from one tile system to another systems. For DNA origami, the limiting reagent is the M13 phage template DNA; all other DNA strands are added in large excess (10-fold to 100-fold). A native PAGE analysis of DNA structures and gel electrophoresis of DNA origami allows a showing that the fully assembled tile should be the slowest-migrating species on native PAGE. Equal amounts of each template and a 10-fold excess of all other DNA strands produced predominantly two species (Lane 1, FIG. 8), making it difficult to assign either species as fully assembled tile. For DNA tile design #1, a 1:1 ratio of templates to all staples, produced a species that migrated much more slowly along with material that did not migrate out of the well (Lane 2, FIG. 8). Looking carefully at the tile design, a large excess of SZ could potentially produce two fragments, one for T1 and another for T2, that could each bind SZ. To test this idea, samples with 0, 0.1, 1, and 2 equivalents of SZ were prepared and analyzed by native PAGE (FIG. 9). Lane 4 in FIG. 9 shows that 1 equivalent of SZ and a 10-fold excess of all other staple strands generated a species that migrated much more slowly than when SZ was in excess. Lane 5, containing a 2-fold excess of SZ over templates, shows the same slow migrating species in addition to the two species observed when SZ was in large excess (Lane 1). Based on these results, experiments with DNA tile design #1 used a 1:1 ratio of templates to SZ.

It is contemplated that the template strands in DNA tile design #1 have identical sequences at the 5′ and 3′ termini. These sequences can be included to enable PCR amplification of the templates. This sequence duplication can lead to formation of unintended structures and redesign of the templates and staples SX and SY to create design #2, For DNA tile design #2, a 1:1 ratio of templates to all staples can have the highest yield of fully assembled tile, and, unlike DNA tile design #1, no DNA may remain in the well. The sequence duplication in design 41 can be responsible for aggregate formation (Lane 2, FIG. 8: equimolar DNA ratios) through crosslinking mediated by staples SX or SY.

As described above, the slowest migrating species observed by native PAGE was assigned to a putative fully assembled tile species. To help support the assignment, tile fragments were prepared, and their migration rates were determined and compared to a commercially available DNA ladder. A single template (T1 & T2 from design #2 fused together) was also used to assemble the tile. These experimental results and the overall reproducibility of native PAGE support the assignment.

Spin-labeled staples are incorporated into the DNA tile (EPR spectra of SL-oligos & final SL-tiles). Multiple methods are available to add spin-labels to the DNA tile. One option is to include the strand(s) with modifications (for attachment chemistry) in the tile assembly process, purify the assembled tile, and then covalently attach the spin label(s). The tiles assemble with alkyne-labeled strand and could be purified. A second option is to spin-label and purify the modified strand(s) and then proceed with the tile assembly process. Adopting this strategy results in stock solutions of purified, spin-labeled strands that can be used to generate a collection of labeled tiles with minimal effort (fewer labeling reactions).

EPR spectra of the spin-labeled 16-mers, S1 to S5. These spectra are characteristic of a spin label attached to a small biomolecule. As a control experiment, the EPR, spectrum of the TEMPO-N3 spin label was monitored before and after exposing it to the annealing procedure for tile assembly. The spectrum intensity decreased significantly (data not shown), indicating that this specific spin label cannot be present on the strand(s) during annealing; other types of labels may be more robust. As an alternative, DNA tiles missing the spin-labeled strand(s) were annealed and the spin-labeled strands) added afterwards. Gel electrophoresis of the pre- and post-addition tiles indicates that an incubation time of 1 h is sufficient to incorporate the missing strand(s). To conserve spin-labeled staple(s), the concentration of the staple(s) was the same as the template strands in the assembly solution. Using this method, tiles containing spin-labeled 16-mers S2 and S4 were prepared and purified by centrifugal filtration.

Nucleic acid tile structure 100 in accordance with embodiments of the present invention has several advantages over previous nucleic acid tile structures. The use of probe oligomers to interact with molecular probes, as disclosed herein, allows molecular probes to be positioned at predetermined locations on nucleic acid tile structure 100 with nanometer-scale accuracy, which reduces the amount of commercially-synthesized DNA required to produce the nanostructure and, thereby, the cost for scale-up and thus has widespread use in, for example, the fields of nanoelectronics, nanomechanical devices, biosensors, programmable/autonomous molecular machines, and molecular computing systems. Nucleic acid tile structures in accordance with embodiments of the present invention provide spatial control of DNA nanofabrication and does so at significantly lower molar cost.

Nucleic acid tile structure in accordance with one or more embodiments of the present invention can be adapted to a variety of configurations. It is thought that nucleic acid tile structure in accordance with various embodiments of the present invention and many of its attendant advantages will be understood from the foregoing description and it will be apparent that various changes may be made without departing from the spirit and scope of the invention or sacrificing all its material advantages, the form hereinbefore described being merely a preferred or exemplary embodiment thereof.

Those familiar with the art will understand that embodiments of the invention may be employed, for various specific purposes, without departing from the essential substance thereof. The description of any one embodiment given above is intended to illustrate an example rather than to limit the invention. This above description is not intended to indicate that any one embodiment is necessarily preferred over any other one for all purposes, or to limit the scope of the invention by describing any such embodiment, which invention scope is intended to be determined by the claims, properly construed, including all subject matter encompassed by the doctrine of equivalents as properly applied to the claims.

What is claimed is:

1. A nucleic acid tile structure of a predetermined size comprising:
a first template oligonucleotides having a first length comprising a first, a second, a third, a fourth, a fifth, a sixth, and a seventh template domains, wherein the second, the fifth and the sixth template domains are positioned at predetermined locations on the first template oligonucleotides;
a second template oligonucleotides having a second length comprising an eighth, a ninth, a tenth, an eleventh, a twelfth, a thirteenth, and a fourteenth template domains, wherein the ninth and the twelfth template domains are positioned at predetermined locations on the second template oligonucleotides, wherein the predetermined size of the nucleic acid tile structure is determined by the first and the second lengths of the first and the second template oligonucleotides;
a first staple oligonucleotides comprising a first, a second and a third staple domains, wherein the first staple domain hybridizes to the thirteenth template domain, wherein the second staple domain hybridizes to the first template domain, wherein the third staple domain hybridizes to the seventh template domain, wherein the hybridizing the first, the second and the third staple domains to the first, the seventh and the thirteenth template domains positions the hybridized first staple oligonucleotide to crossover from the first template oligonucleotide to the second template oligonucleotide and from a first portion to a second portion of the first template oligonucleotide;
a second staple oligonucleotides comprising a fourth and a fifth staple domains, wherein the fourth staple domain hybridizes to the eighth template domain, wherein the fifth staple domain hybridizes to the fourteenth template domain, wherein the hybridizing the fourth and the fifth staple domains to the eighth and the fourteenth template domains positions the hybridized second staple oligonucleotide to crossover from a first portion to a second portion of the second template oligonucleotide;
a third staple oligonucleotides comprising a sixth, a seventh, an eighth and a ninth staple domains, wherein the sixth staple domain hybridizes to the fourth template domain, wherein the seventh staple domain hybridizes to the third template domain, wherein the eighth staple domain hybridizes to the eleventh template domain, wherein the ninth staple domain hybridizes to the tenth template domain, wherein hybridizing the sixth, the seventh, the eighth and the ninth staple oligonucleotides hybridizing to the third, the fourth, the tenth, and the eleventh template domains positions the hybridized third staple oligonucleotide to crossover from the first template oligonucleotide to the second template oligonucleotide and from a third portion to a fourth portion of the first template oligonucleotide and from a third portion to a fourth portion of the second template oligonucleotide;
a first probe oligonucleotide comprising nucleotide sequence complementary to the sixth template domain, wherein the first probe oligonucleotide hybridizes to the sixth template domain;
a second probe oligonucleotide comprising nucleotide sequence complementary to the fifth template domain, wherein the second probe oligonucleotide hybridizes to the fifth template domain;
a third probe oligonucleotide comprising nucleotide sequence complementary to the second template domain, wherein the third probe oligonucleotide hybridizes to the second template domain;
a fourth probe oligonucleotide comprising nucleotide sequence complementary to the twelfth template domain, wherein the fourth probe oligonucleotide hybridizes to the twelfth template domain; and
a fifth probe oligonucleotide comprising nucleotide sequence complementary to the ninth template domain, wherein the fifth probe oligonucleotide hybridizes to the ninth template domain.

2. The nucleic acid tile structure of claim 1, wherein at least one of the first, the second, the third, the fourth and the fifth probe oligonucleotides further comprises a binding site.

3. The nucleic acid tile structure of claim 2, wherein the binding site is 5-ethynyl-2′-deoxyuridine nucleotide.

4. The nucleic acid tile structure of claim 1, wherein the crossovers of the first, the second, and the third staple oligonucleotide are positioned to fold the first and the second template nucleotide into a predetermined two-dimensional shape.

5. The nucleic acid tile structure of claim 1, wherein the crossovers of the first, the second, and the third staple oligonucleotide are positioned to fold the first and the second template nucleotide into a rectangular structure having a length of about 10 nm and a width of about 8 nm.

6. The nucleic acid tile structure of claim 1, further comprising a plurality of molecular probes, wherein at least one of the plurality of the molecular probes is bound to at least one of the first, the second, the third, the fourth and the fifth probe oligonucleotides.

7. The nucleic acid tile structure of claim 6, wherein the at least one of the plurality of the molecular probes is bound to the at least one of the first, the second, the third, the fourth and the fifth probe oligonucleotides using a covalent bond.

8. The nucleic acid tile structure of claim 6, wherein the plurality of the molecular probes is selected from the group consisting of DNA, RNA, polypeptides, lipids, carbohydrates, other organic molecules, inorganic molecules and metallic particles, ferromagnetic particles, and quantum dots.

9. The nucleic acid tile structure of claim 6, wherein the at least one of the plurality of the molecular probes is a spin label.

10. The nucleic acid tile structure of claim 1, wherein distances between the first, the second, the third, the fourth and the fifth probe oligonucleotides are from about 3 nm to about 9 nm.

11. A nucleic acid tile structure of a predetermined size comprising:
a first template oligonucleotides having a first length comprising a first, a second, a third, a fourth, a fifth, a sixth, and a seventh template domains, wherein the second, the fifth and the sixth template domains are positioned at predetermined locations on the first template oligonucleotides;
a second template oligonucleotides having a second length comprising an eighth, a ninth, a tenth, an eleventh, a twelfth, a thirteenth, and a fourteenth template domains, wherein the predetermined size of the nucleic acid tile structure is determined by the first and the second lengths of the first and the second template oligonucleotides, wherein the ninth and the twelfth template domains are positioned at predetermined locations on the second template oligonucleotides;
a first staple oligonucleotides comprising a first, a second and a third staple domains, wherein the first staple domain hybridizes to the thirteenth template domain, wherein the second staple domain hybridizes to the first template domain, wherein the third staple domain hybridizes to the seventh template domain;
a second staple oligonucleotides comprising a fourth and a fifth staple domains, wherein the fourth staple domain hybridizes to the eighth template domain, wherein the fifth staple domain hybridizes to the fourteenth template domain;
a third staple oligonucleotides comprising a sixth, a seventh, an eighth and a ninth staple domains, wherein the sixth staple domain hybridizes to the fourth template domain, wherein the seventh staple domain hybridizes to the third template domain, wherein the eighth staple domain hybridizes to the eleventh template domain, wherein the ninth staple domain hybridizes to the tenth template domain;
a first probe oligonucleotide hybridized to the sixth template domain, wherein the first probe oligonucleotide further comprises a 5-ethynyl-2′-deoxyuridine nucleotide;
a second probe oligonucleotide hybridized to the fifth template domain;
a third probe oligonucleotide hybridized to the second template domain;
a fourth probe oligonucleotide hybridized to the twelfth template domain;
a fifth probe oligonucleotide hybridized to the ninth template domain; and
a first molecular probe bound to the 5-ethynyl-2′-deoxyuridine nucleotide of the first probe oligonucleotide.

12. The nucleic acid tile structure of claim 11, wherein the first molecular probe is a spin label.

13. The nucleic acid tile structure of claim 11, further comprising a second, a third, a fourth and a fifth molecular probes bound to the second, the third, the fourth and the fifth probe oligonucleotides.

14. The nucleic acid structure of claim 12, wherein the second, the third, the fourth and the fifth molecular probes is selected from the group consisting of DNA, RNA, polypeptides, lipids, carbohydrates, other organic molecules, inorganic molecules and metallic particles, ferromagnetic particles, and quantum dots.

15. The nucleic acid tile structure of claim 1, wherein the first, the second, and the third staple oligonucleotide hybridizing to the first and the second template oligonucleotide folds the first and the second template nucleotide into a rectangular shape.

16. A method for making a non-naturally occurring nucleic acid tile structure, said method comprising:
providing a first template oligonucleotide comprising a first, a second, a third, a fourth, a fifth, a sixth, and a seventh template domains;
providing a second template oligonucleotides comprising an eighth, a ninth, a tenth, an eleventh, a twelfth, a thirteenth, and a fourteenth template domains;
providing a first staple oligonucleotides comprising a first, a second and a third staple domains;
providing a second staple oligonucleotides comprising a fourth and a fifth staple domains;
mixing the first template oligonucleotide, the second template oligonucleotide, the first staple oligonucleotide and the second oligonucleotide to form a first mixture;
annealing the first mixture to hybridize the first staple domain to the thirteenth template domain, the second staple domain to the first template domain, the third staple domain to the seventh template domain, the fourth staple domain to the eighth template domain, and the fifth staple domain to the fourteenth template domain;
providing a third staple oligonucleotides comprising a sixth, a seventh, an eighth and a ninth staple domains;
mixing the third staple oligonucleotide with the annealed first mixture to form a second mixture;
annealing the second mixture to hybridize the sixth staple domain to the fourth template domain, the seventh staple domain to the third template domain, the eighth staple domain to the eleventh template domain, and the ninth staple domain to the tenth template domain;
providing a first, a second, a third, a fourth and a fifth probe oligonucleotides;
mixing the first, the second, the third, the fourth and the fifth probe oligonucleotides with the annealed second mixture to form a third mixture;
annealing the third mixture to hybridize the first probe oligonucleotide to the sixth template domain, the second probe oligonucleotide to the fifth template domain, the third probe oligonucleotide to the second template domain, the fourth probe oligonucleotide to the twelfth template domain, and the fifth probe oligonucleotide to the ninth template domain; and
mixing a plurality of molecular probes to annealed third mixture to bind at least one of the plurality of the molecular probes to at least one of the first, the second, the third, the fourth and the fifth probe oligonucleotides.

17. The method of claim 16, wherein the first probe oligonucleotide further comprises a 5-ethynyl-2′-deoxyuridine nucleotide.

18. The method of claim 17, wherein the at least one of the plurality of the molecular probes is bound to the 5-ethynyl-2′-deoxyuridine nucleotide of the first probe oligonucleotide.

19. The method of claim 18, wherein the at least one of the plurality of the molecular probes bound to the 5-ethynyl-2′-deoxyuridine nucleotide is a spin label.

20. The method of claim 16, wherein the second, the fifth and the sixth template domains are positioned at predetermined locations on the first template oligonucleotides, and wherein the ninth and the twelfth template domains are positioned at predetermined locations on the second template oligonucleotides.
